(19) United States
(12) Patent Application Publication
(10) Pub. No.: US 2021/0236642 A1
(43) Pub. Date: Aug. 5, 2021

(54) HAEMOSTATIC GEL COMPOSITION AND ITS PROCESS OF PREPARATION

(71) Applicants: Srinivas Reddy MALE, Hyderabad (IN); Praveen Kumar MANDAPALL, Hyderabad (IN); Sunny Babarao WANKHADE, Hyderabad (IN); Shantaram Laxman PAWAR, Hyderabad (IN); Azista Industries Pvt Ltd, Hyderabad (IN)

(72) Inventors: Srinivas Reddy Male, Hyderabad (IN); Praveen Kumar Mandapalli, Hyderabad (IN); Sunny Babarao Wankhade, Hyderabad (IN); Shantaram Laxman Pawar, Hyderabad (IN)

(21) Appl. No.: 17/263,041

(22) PCT Filed: Jul. 26, 2019
(86) PCT No.: PCT/IB2019/056385

§ 371 Date: Jan. 25, 2021

(30) Foreign Application Priority Data
Jul. 26, 2018 (IN) 201841028114

(51) Int. Cl. A61K 47/36; A61K 47/42; A61L 26/00

(52) U.S. Cl. CPC A61K 47/36; A61K 47/42; A61L 2400/04; A61L 26/0052; A61L 26/008

(57) ABSTRACT

The present invention relates to the haemostatic gel composition comprising polysaccharide, protein and carbohydrate in a single phase. The present invention specifically relates to the haemostatic gel composition comprising chitosan as polysaccharide, gelatin as protein and starch as carbohydrate in a single phase. The present invention more specifically relates to efficient process for preparation of hemostatic gel, wherein the process comprising steps of dispersing, dissolving, filling, sterilizing and packaging.

### FIELD OF THE INVENTION

The present invention relates to a haemostatic gel composition comprising polysaccharide, protein and carbohydrate in a single phase.

The present invention also relates to efficient process for the preparation of haemostatic gel, wherein process comprising steps of dispersing, dissolving, filling, sterilizing and packaging.

### BACKGROUND OF THE INVENTION

Bleeding is one of the major cause for the death in traumatic injuries. It is important to stop bleeding and facilitate faster clotting than the human body would achieve clotting on its own. To achieve blood clotting more quickly, medical practitioner sometimes use hemostatic agents. These haemostatic agents may promote clotting and thereby stop or control bleeding.

A variety of hemostatic agents are used with varying efficacy and varying degrees of evidence in favour of their benefits. Some polysaccharides and proteins can also be used as haemostatic agents because of its nontoxic nature to humans and easily absorbed by the body. These agents may promote stable and rapid clotting because of its long chain length and branching in structure.

Several polysaccharide and protein based hemostatic agents have been approved for use in the last few years, including chitosan-based agents, gelatin based agents and starch based agents. These agents have already shown great promise in controlling major haemorrhage conditions in the pre-hospital setting and in animal models.

### Gelatin Based Hemostatic Agents

Gelatin based hemostatic agents are widely used in modern operating rooms and first introduced in the 1940s as Gelfoam. Gelfoam is made of gelatin and is prepared from purified porcine skin. Hemostatic properties of gelatin are not entirely understood, and felt to be more physical rather than related to direct effects on the clotting cascade.

Gelfoam can be used in several ways, either in dry sponge form, moistened with injectable sodium chloride solution, or commonly, saturated with topical purified thrombin. The combined application with topical thrombin is one of the few advancements made with gelatin-based hemostatic agents.

Another major advancement in the field of gelatin-based hemostatic agents came in the development of the product Floseal. Approved for commercial use in the US in 1999, Floseal combines human-derived thrombin with bovine-derived gelatin matrix granules, which are mixed at the time of use.

### Starch Based Hemostatic Agents

Starch based haemostatic agents; especially in powder form has shown to be effective in controlling bleeding conditions. To date, there are no serious product related adverse events known. There are numerous articles published which are dealing with starch based hemostatic agents. The degradation process of starch particles is enzymatically performed by Amylase and Pyrase.

Arista™ AH is a 100% plant based absorbable surgical hemostatic powder derived from purified plant starch. The power of Arista™ AH lies in its Microporous Polysaccharide Hemospheres, a patented blood clotting technology.

Starch acts like a filter which concentrates the blood cells and the proteins such as thrombin, albumin and fibrinogen with a haemostasis occurring in a few minutes.

### Chitosan Based Hemostatic Agents

Chitosan based hemostatic agents are available in different forms including sponges, films, gels, beads and fibres. Chitosan is extracted from crab or shrimp shells. Chitosan is a linear, semi-crystalline polysaccharide composed of (1-4)-2-acetamido-2-deoxyb-D-glucan (N-acetyl D-glucosamine) and (1-4)-2-amino-2-deoxyb-D-glucan (D-glucosamine) units.

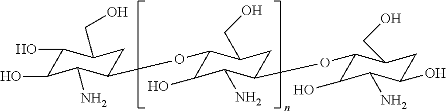

Chitosan has been widely used in various bleeding conditions and injuries for its haemostatic effect. The specific mechanism of action of chitosan remains undiscovered but data suggest about three possible ways to control bleeding: 1) sorption of plasma, 2) erythrocytes coagulation, and 3) platelet adhesion, aggregation and activation.

Chitosan as wound dressing material is used in various types of conditions including haemostasis, pressure sores, diabetic ulcers, leg ulcer, donor sites and graft sites, surgical wounds, skin abrasions and lacerations, 1st and 2nd degree burns, trauma wounds.

Hemcon, Chitoflex, Chitoseal, TraumaStat and Celox are the most commonly used chitosan based hemostatic agents available in market.

The important challenge in the treatment of bleeding condition is adhesive property of physical barrier component of given haemostatic material. If the blood flow is strong enough, haemostasis can be disrupted as premature platelet plugs and fibrin clots may be ruptured in the process. The main problem is due to haemostatic device lacks sufficient adhesion and a partially formed plug or clot disengages from the wound area.

Most of the hemostatic products available in market are suitable only for surface wounds, where pressure must be applied or else pose the risk of releasing clotting factors into the circulatory system. Many types of hemostatic agents come in powder form. Powder can be an inconvenient form of delivery as it is difficult to handle. In addition, powder can be difficult to apply to various areas of the body such as the nose, the gums during oral Surgery, or the back.

CN 105727344 discloses composite hemostatic membrane material comprising a chitosan membrane layer and a starch/gelatin membrane layer.

U.S. Pat. No. 4,532,134 discloses the medical properties of chitosan which include hemostasis, hindered of growth of fibroblast, and improving tissue regeneration.

U.S. Pat. No. 5,612,052 discloses hydrogel-forming, self-solvating, absorbable polyester copolymers capable of selective, segmental association into compliant hydrogels upon contacting an aqueous environment.

U.S. Pat. No. 6,821,331 discloses protein-polysaccharide hydrogel. It also discloses hydrogel which includes two interpenetrating matrices: a first matrix which is an acylated, cross-linked protein matrix, and a second matrix which is an anionic polysaccharide matrix.

Malette et al., The Annals of Thoracic Surgery, 1983, 36(1), 55-58 discloses use of chitosan solution as a hemostatic agent to prevent blood loss from porous vascular grafts and chitosan in some way allows the ingrowth of vascularized smooth supporting an endothelial luminal surface.

Deng et al., Scientific Reports, 7(2699), 1-13 discloses injectable in situ cross-linking chitosan-hyaluronic acid based hydrogels.

None of the prior art references discloses the haemostatic gel comprising chitosan, gelatin and starch in one single phase. However, the inventors of present invention provide haemostatic gel composition comprising chitosan, gelatin and starch. The inventors of present invention also provide an efficient process for preparation of hemostatic gel, wherein the process comprising steps of dispersing, dissolving, filling, sterilizing and packaging.

### OBJECTIVE OF THE INVENTION

The main objective of the present invention is to provide haemostatic gel composition comprising polysaccharide, protein and carbohydrate in a single phase.

Another objective of the present invention is to provide haemostatic gel composition comprising chitosan as polysaccharide, gelatin as protein and starch as carbohydrate.

Another objective of the present invention is to provide a process for preparing haemostatic gel, wherein process comprises preparation of hemostatic gel by dissolving raw materials in water and mix together, filling, sterilization and packaging.

Still another objective of the present invention is to provide a process for preparing haemostatic gel, wherein the prepared hemostatic gel is filled syringe applicator for easy application to internal organs or deep cut or wounds.

### SUMMARY OF THE INVENTION

Accordingly, the present invention provides composition of biocompatible gel useful in facilitating and maintaining hemostasis.

In one embodiment, the present invention relates to haemostatic gel composition comprising natural, biodegradable and biocompatible materials in a single phase.

In one embodiment, the present invention relates to hemostatic gel composition comprising a) one or more polysaccharides, b) one protein, c) one carbohydrate and d) a solvent in a single phase.

In one embodiment, the present invention relates to hemostatic gel composition comprising a) chitosan, b) gelatin, c) starch and d) water in a single phase.

In one embodiment, the present invention relates to hemostatic gel composition comprising:

a) 1% to 20% (w/w) of polysaccharide,

b) 1% to 20% (w/w) of protein,

c) 1% to 20% (w/w) of carbohydrate, and

d) solvent.

In one embodiment, the present invention relates to hemostatic gel composition comprising:

a) 2% to 10% (w/w) of polysaccharide,

b) 2% to 10% (w/w) of protein,

c) 4% to 15% (w/w) of carbohydrate, and

d) solvent.

In one embodiment, the present invention relates to hemostatic gel composition comprising:

a) 2% to 10% (w/w) of chitosan,

b) 2% to 10% (w/w) of gelatin,

c) 4% to 15% (w/w) of starch, and

d) solvent.

Another embodiment of the present invention provides a process for preparing haemostatic gel, wherein the process comprising steps of:

a) dispersing polysaccharide in solvent under stirring and homogenization,

b) dissolving protein in hot solvent under stirring,

c) dissolving carbohydrate in hot solvent under stirring,

d) mixing all the above three phases under stirring to obtain homogenous gel,

e) transferring prepared homogenous gel into syringe applicator, and

f) sterilizing the homogenous gel by autoclave/gamma irradiation.

Another embodiment of the present invention provides a process for preparing chitosan haemostatic gel, wherein the process comprising steps of:

a) dispersing chitosan in water under stirring and homogenization,

b) dissolving gelatin in hot water at 80° C. under stirring,

c) dissolving starch in hot water at 80° C. under stirring,

d) mixing all the above three phases under stirring to obtain homogenous gel,

e) transferring prepared homogenous gel into syringe applicator, and

f) sterilizing the homogenous gel by autoclave/gamma irradiation of final packages sponge by 60 Co source at the doses of 4 kGy and 25 kGy.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

Hemostasis is defined as “the stoppage of bleeding, hemorrhage, or blood flow through a blood vessel or body part.” Hemostatic agents improve hemostasis by improving primary hemostasis, stimulating fibrin formation, or inhibiting fibrinolysis.

### Ideal Characteristics of Hemostatic Agents

(1) being capable of stopping large-vessel arterial and venous bleeding within 2 minutes of application, and the ability to be delivered through a pool of blood when applied;
(2) being ready to use with no requirement for on-scene mixing or pre-application preparation;
(3) being simple to use by the wounded victim, a ‘buddy’ or a medic, with minimal training;
(4) having lightweight and durable properties;
(5) having a minimum 2-year shelf-life, in extreme environmental conditions (ideally at a temperature range of −10° C. to +55° C.);
(6) being safe to use with no risk of further injury to tissues or transmission of infection; and
(7) being inexpensive.

Hemostatic dressings can be classified based on their mechanism of action.

1) Factor concentrators: This class of hemostatic agents work through fast absorption of the water content of blood; consequently, concentration of its cellular and protein components results in clot formation. QuikClot (Z-Medica LLC., Newington, Conn., USA), QuikClot ACS (advanced clotting sponge) (Z-Medica LLC., Newington, Conn., USA), TraumaDex (Medafor Inc, Minneapolis, Minn., USA), and self-expanding hemostatic polymer (Payload Systems Inc., Cambridge, Mass., USA) are examples of this group.
2) Mucoadhesive agents: these agents act through a strong adherence to the tissues, and physically block bleeding from wounds. HemCon (HemCon Medical Technologies Inc. Portland, Oreg., USA) and Celox (Medtrade Products Ltd. Crewe, UK) are the main examples of this group.
3) Procoagulant supplementors: agents placed in this group act mainly through delivering procoagulant factors to the hemorrhagic wound. The dry fibrin sealant dressing (DFSD) is an example of these agents (Trauma Mon. 2016 February; 21(1): e26023)

The invention disclosed herein is a class of biodegradable and biocompatible gel compositions useful in facilitating and maintaining hemostasis. The biocompatible gel composition generally comprises a) polysacharide, b) protein, c) carbohydrate, and d) solvent.

The present invention is to provide an effective process for the preparation of injectable haemostatic gel comprising polysacharide, protein, carbohydrate and solvent in a single phase.

The present invention is to provide an effective process for the preparation of injectable haemostatic gel comprising chitosan as polysacharide, gelatin as protein, starch as carbohydrate and water as solvent in a single phase.

Various properties associated with each component of the biocompatible hemostatic gel compositions may affect the properties of the final product. Properties associated with the selection of raw materials, molecular weight, degree of deacetylation, concentration, bloom strength, viscosity in solution. Final biocompatible hemostatic gel composition properties include viscosity, hemostatic efficacy and pH.

The ideal combination for haemostasis, individually chitosan, gelatin and starch are well known as haemostatic agents and already available in market in different forms. In the present invention, we have developed an ideal formulation in combination of these three materials to form haemostatic gel, which accelerates or enhance or synergies the clot formation very effectively in an emergency or during surgery.

The components of the haemostatic gel composition, the biopolymer chitosan is separately dispersed in water under stirring and subjected for homogenization. In another embodiment, gelatin is separately dissolved in water under stirring at 80° C. temperature. Starch is also separately dissolved in water at 80° C. temperature. Mix all three phases under stirring to obtain homogenous gel. The prepared gel is transferred in to syringe applicator. Gel containing syringe applicator is sterilized by moist heat sterilization (Autoclave) or gamma irradiation.

Manufacturing process for the haemostatic gel.

1. Preparation of Chitosan Dispersion (Phase A)

The chitosan is used to prepare the chitosan dispersion, preferably has a degree of deacetylation in the range of 75-85%. The chitosan dispersion preferably prepared at room temperature by addition of chitosan powder or flakes to water under stirring. Later, chitosan dispersion is homogenized to obtain homogenous dispersion. Preferably, the concentration of chitosan should be in the range of 2-10% (w/w). Preferably, the purified water should be around 20% (w/w) of batch size.

2. Preparation of Gelatin Solution (Phase B)

The gelatin is used to prepare gelatin solution and is prepared by addition of gelatin to hot water under stirring, preferably at 80° C. temperature. Preferably, the concentration of gelatin should be in the range of 2-10% (w/w). Preferably, the purified water should be around 20% (w/w) of batch size.

3. Preparation of Starch Paste (Phase C)

The starch is used to prepare is starch paste and is prepared by addition of starch to hot water under stirring, preferably at 80° C. temperature. Preferably, the concentration of starch should be in the range of 4-15% (w/w). Preferably, the purified water should be around 60% (w/w) of batch size.

4. Preparation of Haemostatic Gel by Mixing 3 Phases

Haemostatic gel is prepared by mixing all the three phase in separate vessel under stirring to obtain homogenous gel.

5. Filling into Syringe Applicator

Finally, the prepared gel is filled into syringe applicator for easy application into deep cuts or wounds.

6. Sterilization

Hemostatic gel filled syringe applicator is sterilized by gamma irradiation at the dose in the range of 4 kGy to 25 kGy or sterilized by moist heat sterilization (autoclave).

Chitosan based formulations especially applicable for wounds or direct contact with blood have to be sterilized. Commonly used sterilization methods include steam sterilization, exposure to dry heat and ethylene oxide or gamma irradiation.

Formulations were developed using different concentrations of chitosan, gelatin and starch. Further, the final product is sterilized by autoclave or gamma irradiation at different doses. The formulations prepared with different variations were evaluated for their description, pH, viscosity, spread ability, bacterial endotoxin test and sterility.

The following examples describes the nature of the invention and are given only for the purpose of illustrating the present invention in more detail and are not limitative and relate to solutions, which have been particularly effective on bench scale and prepared by the process of the present invention.

### Example 1

| S. No. | Ingredient | Concentration (% w/w) |
|---|---|---|
| 1 | Chitosan | 6 |
| 2 | Gelatin | 4 |
| 3 | Starch | 10 |
| 4 | Water | 80 |
| | Total | 100 |

### Example 2

| S. No. | Ingredient | Concentration (% w/w) |
|---|---|---|
| 1 | Chitosan | 2 |
| 2 | Gelatin | 4 |
| 3 | Starch | 10 |
| 4 | Water | 84 |
| | Total | 100 |

### Example 3

| S. No. | Ingredient | Concentration (% w/w) |
|---|---|---|
| 1 | Chitosan | 4 |
| 2 | Gelatin | 4 |
| 3 | Starch | 10 |
| 4 | Water | 82 |
| | Total | 100 |

### Example 4

| S. No. | Ingredient | Concentration (% w/w) |
|---|---|---|
| 1 | Chitosan | 8 |
| 2 | Gelatin | 4 |
| 3 | Starch | 10 |
| 4 | Water | 78 |
| | Total | 100 |

### Example 5

| S. No. | Ingredient | Concentration (% w/w) |
|---|---|---|
| 1 | Chitosan | 10 |
| 2 | Gelatin | 4 |
| 3 | Starch | 10 |
| 4 | Water | 76 |
| | Total | 100 |

### Example 6

| S. No. | Ingredient | Concentration (% w/w) |
|---|---|---|
| 1 | Chitosan | 6 |
| 2 | Gelatin | 2 |
| 3 | Starch | 10 |
| 4 | Water | 82 |
| | Total | 100 |

### Example 7

| S. No. | Ingredient | Concentration (% w/w) |
|---|---|---|
| 1 | Chitosan | 6 |
| 2 | Gelatin | 6 |
| 3 | Starch | 10 |
| 4 | Water | 78 |
| | Total | 100 |

### Example 8

| S. No. | Ingredient | Concentration (% w/w) |
|---|---|---|
| 1 | Chitosan | 6 |
| 2 | Gelatin | 8 |
| 3 | Starch | 10 |
| 4 | Water | 76 |
| | Total | 100 |

### Example 9

| S. No. | Ingredient | Concentration (% w/w) |
|---|---|---|
| 1 | Chitosan | 6 |
| 2 | Gelatin | 10 |
| 3 | Starch | 10 |
| 4 | Water | 74 |
| | Total | 100 |

### Example 10

| S. No. | Ingredient | Concentration (% w/w) |
|---|---|---|
| 1 | Chitosan | 6 |
| 2 | Gelatin | 4 |
| 3 | Starch | 4 |
| 4 | Water | 86 |
| | Total | 100 |

### Example 11

| S. No. | Ingredient | Concentration (% w/w) |
|---|---|---|
| 1 | Chitosan | 6 |
| 2 | Gelatin | 4 |
| 3 | Starch | 6 |
| 4 | Water | 84 |
| | Total | 100 |

### Example 12

| S. No. | Ingredient | Concentration (% w/w) |
|---|---|---|
| 1 | Chitosan | 6 |
| 2 | Gelatin | 4 |
| 3 | Starch | 8 |
| 4 | Water | 82 |
| | Total | 100 |

### Example 13

| S. No. | Ingredient | Concentration (% w/w) |
|---|---|---|
| 1 | Chitosan | 6 |
| 2 | Gelatin | 4 |
| 3 | Starch | 15 |
| 4 | Water | 75 |
| | Total | 100 |

### Manufacturing Process

Required quantity of chitosan was added to batch quantity of purified water under stirring at room temperature to obtain homogenous dispersion (Phase A).

Required quantity of gelatin was added to batch quantity of purified water under stirring at 80° C. temperature to obtain solution (Phase B).

Required quantity of starch was added to batch quantity of purified water under stirring at 80° C. temperature to obtain starch paste (Phase C).

Mix all the three phases under stirring to obtain homogenous haemostatic gel.

The obtained gel was filled into syringe applicator and sterilized autoclave or irradiated with gamma irradiation by 60 Co source at the doses of 4 kGy for sterilization.

### Evaluation Tests for Hemostatic Gel:

Description: Off-white to brown coloured gel filled in syringe applicator.
pH: Hemostatic gel pH was measured using digital pH meter.
Viscosity: Viscosity of the prepared gel was measured using Brookfield viscometer.
Assay: Degree of deacetylation of chitosan was measured titration method.
Sterility test: Sterility testing was performed according to USP general chapter <71>.
Antimicrobial effectiveness testing: Antimicrobial effectiveness testing was performed according to USP General Chapter (51).
Bacterial Endotoxin Test: Bacterial Endotoxin Test was performed for optimized formulation. The testing was performed according to USP General Chapter <85>.

TABLE 1
Specifications for haemostatic gel:

| S. No. | Test | Specification |
|---|---|---|
| 1 | Description | Off-white to brown colored gel |
| 2 | Sterility | To pass the test as per USP |
| 3 | Antimicrobial effectiveness testing | To pass the test as per USP |
| 4 | Bacterial endotoxins | NMT 300 IU/g of chitosan |

The haemostatic gel is prepared as per Example 1 of the present invention is evaluated for the above characters at 40° C./75% RH stability conditions and the data is given below table;

TABLE 2
Stability Condition: 40° C./75% RH

| Tests | Specification | Initial | 3 Month |
|---|---|---|---|
| Description | Off white to brown | Off white to brown | Off white to |
| | coloured gel | colored gel | brown |
| | | | colored gel |
| Sterility | To pass the test | Pass | — |
| | as per USP | | |
| Antimicrobial | To pass the test | Pass | — |
| effectiveness | as per USP | | |
| testing | | | |
| Bacterial | NMT 300 IU/g | Complies | — |
| endotoxins test | of chitosan | | |

We claim:

1. Haemostatic gel composition comprising:
a) one or more polysaccharides,
b) protein,
c) carbohydrate, and
d) solvent
in a single phase.

2. Haemostatic gel composition as claimed in claim 1, wherein the polysaccharide used herein is in the range of 1% to 20% (w/w), specifically in the range of 2% to 10% (w/w).

3. Haemostatic gel composition as claimed in claim 1, wherein the polysaccharide used herein is chitosan.

4. Haemostatic gel composition as claimed in claim 1, wherein the protein used herein is in the range of 1% to 20% (w/w), specifically in the range of 2% to 10% (w/w).

5. Haemostatic gel composition as claimed in claim 1, wherein the protein used herein is gelatin.

6. Haemostatic gel composition as claimed in claim 1, wherein the carbohydrate used herein is in the range of 1% to 20% (w/w), specifically in the range of 4% to 15% (w/w).

7. Haemostatic gel composition as claimed in claim 1, wherein the carbohydrate used herein is starch.

8. Haemostatic gel composition as claimed in claim 1, wherein the solvent used herein is water.

9. Efficient process for the preparation of hemostatic gel, wherein the process comprising steps of dispersing, dissolving, filling, sterilizing and packaging.

10. The process for the preparation of haemostatic gel as claimed in claim 9, wherein the process comprising steps of:
a) dispersing polysaccharide in solvent under stirring and homogenization,
b) dissolving protein in hot solvent under stirring,
c) dissolving carbohydrate in hot solvent under stirring,
d) mixing all the above three phases under stirring to obtain homogenous gel,
e) transferring prepared homogenous gel into syringe applicator, and
f) sterilizing the homogenous gel by autoclave/gamma irradiation.

11. The process for the preparation of haemostatic gel as claimed in claim 10, wherein the process comprising steps of:
a) dispersing chitosan in water under stirring and homogenization,
b) dissolving gelatin in hot water at 80° C. under stirring,
c) dissolving starch in hot water at 80° C. under stirring,
d) mixing all the above three phases under stirring to obtain homogenous gel,
e) transferring prepared homogenous gel into syringe applicator, and
f) sterilizing the homogenous gel by autoclave/gamma irradiation of final packages sponge by 60 Co source at the doses of 4 kGy and 25 kGy.
